(19) United States
(12) Patent Application Publication
(10) Pub. No.: US 2009/0247569 A1
(43) Pub. Date: Oct. 1, 2009

(54) Process for Preparing Clopidogrel Bisulphate

(76) Inventors: Claude Singer, Kfar Saba (IL); Basem Masarwa, Taibe (IL); Greta Sterimbaum, Rishon-Lezion (IL); Paola Daverio, Milano (IT); Eran Turgeman, Herzelia (IL)

Correspondence Address: KENYON & KENYON LLP, ONE BROADWAY, NEW YORK, NY, 10004, US

(21) Appl. No.: 12/083,128

(22) PCT Filed: Aug. 3, 2007
(86) PCT No.: PCT/US07/17324

§ 371 Date: Jan. 27, 2009

Related U.S. Application Data
(60) Provisional application No. 60/835,551, filed on Aug. 3, 2006.
(60) Provisional application No. 60/858,127, filed on Nov. 9, 2006.
(60) Provisional application No. 60/877,987, filed on Dec. 28, 2006.

(51) Int. Cl. A61K 31/4743; C07D 491/02

(52) U.S. Cl. 514/301; 546/114

(57) ABSTRACT

Provided are processes for the preparation of clopidogrel bisulphate Form I.

### CROSS REFERENCE TO RELATED APPLICATIONS

The present application claims the benefit of the following U.S. Provisional Patent Application Nos.: 60/835,551, filed Aug. 3, 2006, 60/858,127, filed Nov. 9, 2006, and 60/877,987 filed Dec. 28, 2006. The contents of the applications are incorporated herein by reference.

### FIELD OF THE INVENTION

The invention encompasses improved methods for the preparation Clopidogrel Bisulphate and especially for the preparation for the polymorphic form I of this compound.

### BACKGROUND OF THE INVENTION

Methyl(+)-(S)-α-(2-Chlorophenyl)-4,5,6,7-tetrahydro[3,2-c]pyridine-5-acetate sulphate of the following formula:

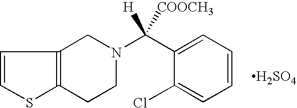

known as Clopidogrel is an inhibitor of induced platelet aggregation which act by inhibiting the binding of adenosine diphosphate to its receptor. Clopidogrel is metabolized by the liver into active form. Its antiplatelet activity is extended in that it stops any platelet activity even up to ten days after administration. Clopidogrel's platelet inhibiting activity makes it an effective drug for reducing the incidence of ischemic strokes, heart attacks or claudication due to vascular diseases such as atherosclerosis. By inhibiting platelet aggregation, clopidogrel reduces the chance of arterial blockage, thus preventing strokes and heart attacks.

Clopidogrel is administered as its hydrogensulfate (syn. bisulfate) salt. Clopidogrel hydrogensulfate has an empirical formula of C₁₆H₁₆ClNO₂S.H₂SO₄. It is currently being marketed as PLAVIX® tablets, which contain about 98 mg clopidogrel hydrogensulfate, which is the equivalent of 75 mg clopidogrel base. PLAVIX® is a white to off-white powder that is practically insoluble in water at neutral pH but highly soluble at acidic pH. It dissolves freely in methanol, somewhat in methylene chloride, and poorly in ethyl ether.

International Publication No. WO 99/65915 discloses two polymorphs of clopidogrel hydrogensulfate, referred to as Forms I and II, though Form I is originally disclosed in EP 281459. According WO '915, Form I has a PXRD pattern with peaks at 9.2, 10.9, 15.2, 17.9, 18.5, 20.6, 23.0, 23.2, 23.4 and 25.5±0.2 degrees two theta. Both forms are crystallized from acetone under different conditions. WO '915 discloses that Form II of Clopidogrel Bisulphate is thermodynamically more stable then Form I. This creates a constant drive in discovering reliable solvents/mixtures for the preparation of Clopidogrel Bisulphate Form I where the spontaneous transformation into Form II can be avoided.

U.S. Pat. No. 6,767,913 discloses new forms III, IV, V and amorphous Clopidogrel Bisulphate, and processes for their preparation.

US publication no. 2006/0041136 discloses the preparation of Form I from Clopidogrel base or Clopidogrel Bisulphate in alcohols or their esters.

US publication no. 2006/0047121 discloses the precipitation of Clopidogrel Bisulphate Form I by dissolving Clopidogrel Bisulphate Form II in a C₁-C₅ carboxylic acid and by precipitating in the presence of an aliphatic or cyclic ether.

Processes for preparation of Clopidogrel Bisulphate are also provided in WO2004/048385 and WO2005/016931.

The present invention relates to the solid state physical properties of clopidogrel bisulfate prepared by any of these or other methods. These properties can be influenced by controlling the conditions under which clopidogrel bisulfate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

### SUMMARY OF THE INVENTION

The present invention provides a process for preparing Clopidogrel Bisulphate comprising: dissolving Clopidogrel base in an organic solvent selected from the group consisting of: C₃-C₈ ether, C₄-C₆ ketone and C₆-C₁₂ aromatic hydrocarbon; and combining the solution with a sulfuric acid, wherein the temperature during the process is of below about 40° C.

The present invention provides a process for preparing clopidogrel Bisulphate Form I comprising: dissolving Clopidogrel base, an organic solvent selected from the group consisting of an C₄-C₅ ketone and C₆-C₁₂ aromatic hydrocarbon to obtain a solution; and adding sulfuric acid to the solution to obtain clopidogrel Bisulphate Form I.

The present invention provides a process for preparing Clopidogrel Bisulphate comprising: combining Clopidogrel Bisulphate, MIBK and Clopidogrel base to obtain a suspension, and adding H₂SO₄ to the suspension, wherein the process is performed at a temperature of about 10° C. to about −20° C. and the Clopidogrel Bisulphate is an amount of at least about 10% weight/weight from the obtained Clopidogrel Bisulphate.

The present invention provides a process for preparing Clopidogrel Bisulphate comprising: combining Clopidogrel base, MIBK and surfactant to obtain a solution, and adding H₂SO₄, wherein the process is performed at a temperature of about 15° C. to about −15° C. Preferably, the Clopidogrel Bisulphate is Clopidogrel Bisulphate Form I.

The present invention provides a process for preparing clopidogrel Bisulphate Form I comprising dissolving Clopidogrel base in MTBE (methyl-t-butyl-ether); cooling; adding formic acid or acetic acid to obtain a cooled solution; and adding the cooled solution to a mixture of sulfuric acid and MTBE at a temperature less than about 40° C. to obtain Clopidogrel Bisulphate.

### DETAILED DESCRIPTION OF THE INVENTION

As used herein, the term “surfactant” refers to an agent that is capable of reducing the surface tension of liquid.

As used herein Form I refers to Form I of clopidogrel hydrogen sulfate disclosed in WO 99/65915, which has a PXRD pattern with peaks at 9.2, 10.9, 15.2, 17.9, 18.5, 20.6, 23.0, 23.2, 23.4 and 25.5±0.2 degrees two theta.

We have found that Form I can be prepared directly from an antisolvent or in a mixture of solvent/antisolvent.

The present invention provides a process for preparing clopidogrel bisulphate Form I comprising: dissolving Clopidogrel base, an organic solvent selected from the group consisting of: C₃-C₈ ether, C₄-C₆ ketone and C₆-C₁₂ aromatic hydrocarbon; adding a sulfuric acid to obtain clopidogrel bisulphate. The clopidogrel bisulphate is preferably isolated. The aromatic hydrocarbon is a liquid at room temperature, i.e., a C₆-C₁₂ hydrocarbon, preferably a C₆ to C₈ hydrocarbon. The C₄-C₆ ketone can be a C₄-C₅ ketone Preferably the sulfuric acid is added at a temperature below about 40° C.

The organic solvent may be one of cyclohexanone, MIBK (methyl-iso-butyl ketone), diethyl ether, methyl t-butyl ether (MTBE), toluene, pentanol, or tetrahydrofuran (THF).

The process can be carried out by dissolving clopidogrel base in one of the above solvents. The ratio of Clopidogrel base to solvent is preferably about 5 to about 20 Clopidogrel/solvent (g/ml). The solution is then combined with sulfuric acid. Preferably the sulfuric acid is concentrated sulfuric acid, i.e., about a 98% solution in water. Preferably, the sulfuric acid is combined with the solution at a temperature of about −20° C. to about 40° C., more preferably, at about −10° C. to about 15° C. Optionally, the sulfuric acid is added to the solution. Preferably, the sulfuric acid is added dropwise to the solution. Preferably, the sulfuric acid is added dropwise. Preferably, the sulfuric acid is added in a period of time of about 0.5 hour to about 5 hours, more preferably about 1 hour. The addition of sulfuric acid results in precipitation of the salt.

Optionally, when the organic solvent is MTBE, methanol is added to the solution of Clopidogrel base prior to combining the solution of Clopidogrel base with the sulfuric acid. Preferably, the Clopidogrel base is first combined with MTBE and thereafter methanol is added.

Preferably, when the organic solvent is MTBE, the solution of Clopidogrel base in MTBE is added to the sulfuric acid.

Optionally, when the organic solvent is MTBE, the process comprises: dissolving Clopidogrel base in MTBE; cooling; adding formic acid or acetic acid to obtain a cooled solution; and adding the cooled solution to a mixture of sulfuric acid and MTBE at a temperature less than about 40° C. to obtain Clopidogrel Bisulphate. Preferably, the solution of Clopidogrel base and MTBE is cooled to a temperature of about −10° C. to about 0° C.

After combining the sulfuric acid with the solution or solvent in any of the above processes, a suspension comprising Clopidogrel Bisulphate salt is obtained. Preferably, the suspension is stirred for about 1 hour to about 70 hours, more preferably, for about 4 hours to about 24 hours.

In one embodiment the process comprises: combining Clopidogrel Bisulphate, MIBK and Clopidogrel base to obtain a suspension, and adding H₂SO₄ to the suspension, wherein the process is at a temperature of about 10° C. to about −20° C. and the Clopidogrel Bisulphate is an amount of at least about 10% weight/weight from the obtained Clopidogrel Bisulphate. Preferably, the temperature during the process is about −10° C. Preferably, the Clopidogrel Bisulphate and MIBK are first combined to obtain a suspension and Clopidogrel base dissolved in MIBK is then added to the suspension. Preferably, the Clopidogrel Bisulphate is present in an amount of about 50%. Preferably, the H₂SO₄ is added dropwise. Preferably, after the addition of the H₂SO₄, a suspension is obtained. Preferably, the suspension is stirred for about 0.5 hour to about 5 hours, more preferably, for about 35 minutes.

In another embodiment the process comprises combining Clopidogrel base, MIBK and surfactant to obtain a solution, and adding H₂SO₄, wherein the process is at a temperature of about 15° C. to about −15° C. Preferably, the temperature during the process is about 5° C. Preferably, prior to the H₂SO₄ addition, the solution is seeded with Clopidogrel Bisulphate. Preferably, the surfactant is selected from the group consisting of: TWEEN® polysorbate and Sodium Lauryl Sulfate (SLS). Preferably, the H₂SO₄ is added dropwise. Preferably, after the addition of the H₂SO₄, a suspension is obtained. Preferably, the suspension is stirred for about 12 hours to about 48 hours, more preferably, for about 24 hours.

The salt from the suspension can then be recovered, such as by filtration. Preferably, the filtration is carried out under a temperature of about −10° C. to about 30° C., more preferably, at a temperature of about 10° C. to about 30° C. Preferably, the recovered Clopidogrel Bisulphate Form I is further dried. Preferably, the drying is under vacuum (pressure of less than about 100 mmHg) at a temperature of about 30° C. to about 40° C.

The Clopidogrel base used in any of the above processes can be prepared by dissolving Clopidogrel camphorsulphonate salt in a mixture of water and MIBK (methyl-isobutyl ketone). An alkali metal or alkaline earth metal base, such as a hydroxide or a carbonate can be added to the solution. In one embodiment, sodium or potassium hydroxide is added to the solution to obtain a pH of about 2-3. NaHCO₃ is then added to reach a pH of about 8. Due to the exothermic nature of the reaction, the reaction mixture can be cooled during the reaction to maintain a temperature of about 25 to about 30° C. The phases of the resulting 2 phase reaction mixture can then be separated and the organic phase washed with water. The reaction mixture can then be concentrated under reduced pressure, elevated temperature, or a combination of both.

The Clopidogrel base may also be prepared by the process disclosed in WO 99/65915, which process is incorporated herein by reference.

Optionally, the concentrated reaction mixture containing Clopidogrel base is distilled prior to the addition of the sulfuric acid. The distillation can also be carried out until obtaining dry Clopidogrel base. When the distillation proceeds until obtaining dry Clopidogrel base, additional amount of organic solvent, as described above, is added. In some instances the organic solvent and the water for an azeotrope during distillation.

The processes of the present invention may be used for industrial scale applications, and the obtained product may be used for additional industrial scale applications.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to limit its scope in any way.

### EXAMPLES

### Example 1

### A process for Preparing Clopidogrel Bisulphate Form I Based on WO 99/65915

In a 1 L three-necked round bottom flask equipped with a mechanical stirrer, a condenser and thermometer 100 g of Clopidogrel Camphorsulphonate salt is dissolved in 200 ml water and 300 ml MIBK is charged. ˜15.5 g of NaOH solution 47% is added in order to reach pH 2-3. Further ˜0.75 g NaHCO₃ is added in order to reach pH 7.9. During the pH correction cooling is applied in order to maintain the mixture at 25-30° C. The phases are separated and the organic phase is washed with water. The solvent is concentrated under vacuum at max. 40° C. until 200 ml Clopidogrel base solution is left in the flask.

The 200 ml solution is diluted with 500 ml MIBK and cooled to 10° C. While maintaining this temperature 14.9 g of sulfuric acid 98% is added drop wise. During the addition a solid is formed which adheres to the stirrer and/all to the reactor wall. After 3-4 h at the same temperature the precipitated solid acquires a powder aspect and is filtered under nitrogen and dried 1 in vacuum at 30-40° C. Yield 70% of Form I Clopidogrel Bisulphate

### Example 2

### A Process for Preparing Clopidogrel Bisulphate Form I

Clopidogrel base is prepared similar to example 1 using as organic solvent Ethyl Acetate and evaporating to dryness. In a 1 L three-necked round bottom flask equipped with a mechanical stirrer and thermometer 101.3 g of Clopidogrel base from the previous step is dissolved in 650 mL of THF and at 25-30° C. 31 g of sulfuric acid 98% is added drop wise at constant temperature. After 5 h stirring at 25-30° C. the product is filtered and dried as in example 1. 125 g (95% yield) of Clopidogrel Bisulphate Form I is recovered.

### Example 3

### A process for Preparing Clopidogrel Bisulphate Form I

Clopidogrel base is prepared similar to example 1 using as organic solvent Toluene and evaporating to dryness.

In a 0.25 L three-necked round bottom flask equipped with a mechanical stirrer, a condenser and thermometer 15 g of Clopidogrel base from the previous step is dissolved in 90 mL of Cyclohexanone. The solution is cooled to −10° C. and 4.8 g sulfuric acid 98% is added drop wise. Stirring was continued 17 h at −10° C. with seeding of Form I then 72 h at 25° C. The product was filtered washed with 9 mL of Cyclohexanone and dried at vacuum at 30-40° C. Yield 78% of Clopidogrel Bisulphate Form I.

### Example 4

### A Process for Preparing Clopidogrel Bisulphate Form I

Clopidogrel base is prepared similar to example 1 using as organic solvent Toluene and evaporating to dryness.

In a 0.25 L three-necked round bottom flask equipped with a mechanical stirrer, a condenser and thermometer 15 g of Clopidogrel base from the previous step is dissolved in 150 mL of MTBE and cooled to −10° C. Sulfuric acid is added drop wise maintaining the same temperature. The mixture is heated to −10° C. seeded once, then heated to 30° C. and seeded again during ˜1 h. At 30° C. the mass is maintained for 17 h. During this time the initially adherent solid transforms into a powder which is filtered and dried at 30° C. in vacuum. 15.4 g (78.6% yield) of Clopidogrel Bisulphate Form I is recovered.

### Example 5

### A Process for Preparing Clopidogrel Bisulphate Form I

Clopidogrel base is prepared similar to example 1 using as organic solvent MTBE and evaporating to dryness.

In a 10 L three-necked glass reactor equipped with a mechanical stirrer and thermometer 482 g of Clopidogrel base from the previous step is dissolved in 7.5 L of MTBE and discharged into a storage vessel. Into the same 10 L reactor 4.7 L MTBE and 0.12 L Methanol is added. The mixture is cooled to −8° C. and 146.5 g of H₂SO₄ 98% is added without exceeding −5° C. In continuation the solution of Clopidogrel base in MTBE is added under mixing without exceeding −5° C. during ˜1 h. The formed precipitate is maintained under mixing for 3 h at the same temperature, and then heated to 25° C. At 25° C. the suspension is mixed for 15 h, then filtered, washed and dried under vacuum at 35-40° C. 534 g was Clopidogrel Bisulphate Form I is obtained (85% yield).

### Example 6

### A Process for Preparing Clopidogrel Bisulphate Form I

Clopidogrel base is prepared similar to example 1 using as organic solvent MTBE and evaporating to dryness.

In a 10 L three-necked glass reactor equipped with a mechanical stirrer and thermometer 7.34 L of MTBE is added and cooled to −10° C. Under cooling 223 g H2SO4 98% is added without exceeding −1° C. In a separate 3 L reactor 734 g Clopidogrel base is dissolved in 1.47 L of MTBE and cooled under mixing to −5° C. After −5° C. was reached 1.84 L of Acetic Acid is added and the cold solution is added during 1 h to the mixture of H₂SO₄ and MTBE without exceeding 0° C. The mass is maintained at 0° C. for another 3 h, heated to 30° C. and maintained >20 h at this temperature. After filtration and drying at 35-40° 804 g Clopidogrel Bisulphate Form I is obtained (yield 84%).

### Example 7

### A Process for Preparing Clopidogrel Bisulphate Form I

In a 10 L three-necked round bottom flask equipped with a mechanical stirrer, a condenser and thermometer 1 kg of Clopidogrel Camphorsulphonate salt is dissolved in 2 L water and 3 L MIBK is charged. ˜93.5 g of NaOH solution 47% is added in order to reach pH 2-3. Further ˜88.2 g NaHCO₃ is added in order to reach pH 7.5. During the pH correction, the two-phase mixture is cooled to maintain a temperature of 25-30° C. The phases are separated and the organic phase is washed with 2.5 L water. The solvent is concentrated to dryness under vacuum at a maximum of 40° C. The residue is further dissolved in 6.2 L of MIBK and kept as stock solution.

In a separate 10 L three-necked glass reactor equipped with a mechanical stirrer and thermometer 100 g of Clopidogrel Bisulphate is suspended in 2.1 L MIBK and cooled to ˜(−10° C.). 0.67 L of the stock solution of Clopidogrel base is charged into a 3L reactor, cooled to −10° C. and added to the suspension of Clopidogrel Bisulphate in MICK. 17.6 g H₂SO₄ 98% is then added to the suspension, while maintaining the temperature of the suspension at ˜−10° C. The suspension is then mixed for 35 minutes.

A new portion of 1.34 L of the stock solution of Clopidogrel base is charged into the 3 L reactor, cooled to −10° C., and added to the suspension of Clopidogrel Bisulphate into the 10 L reactor. 35.2 g of H₂SO₄ 98% is then added to the suspension, while maintaining the temperature of the suspension at ˜−10° C. The suspension is then further mixed for 35 minutes.

The remaining stock solution is added to the 10 L reactor and 103.7 g H₂SO₄ 98% is charged, while maintaining the temperature at −10° C. The suspension is further stirred for 17 h at −10° C., filtered, and dried. Yield 70% of Form I Clopidogrel Bisulphate.

### Example 8

### A Process for Preparing Clopidogrel Bisulphate Form I

Clopidogrel base is prepared starting from 1 kg of Clopidogrel Camphorsulphonate similar to example 7 using as organic solvent MIBK and evaporating to dryness.

In a 10 L three-necked glass reactor equipped with a mechanical stirrer and thermometer 6.3 L of MIBK is charged and the reactor is cooled to 5° C. The solution is seeded with 0.25 g Clopidogrel Bisulphate and 50 g of the well known commercial surfactant TEEN® 80 is added. 580 g of Clopidogrel base from the previous step is dissolved and 177 g of H₂SO₄ 98% is added drop wise maintaining the temperature at <5° C. The formed suspension is mixed for 24 h at 5-8° C. and is further discharged on a filter. After washing with 0.5 L MIBK the product is dried under vacuum. 618.7 g dried Clopidogrel Bisulphate form I is obtained.

1. A process for preparing clopidogrel Bisulphate Form I comprising: dissolving Clopidogrel base in an organic solvent selected from the group consisting of an C₆ ketone and C₆-C₁₂ aromatic hydrocarbon to obtain a solution; and adding sulfuric acid to the solution to obtain clopidogrel Bisulphate Form I.

2. The process of claim 1, wherein sulfuric acid is added at a temperature below about 40° C.

3. The process of claim 1, wherein the organic solvent is selected from the group consisting of: toluene, pentanol, MTBE (methyl-t-butyl-ether), and cyclohexanone.

4. The process of claim 3, wherein the solvent is toluene.

5. The process of claim 3, wherein the solvent is pentanol.

6. The process of claim 3, wherein the solvent is cyclohexanone.

7. The process of claim 3, wherein the solvent is MTBE (methyl-t-butyl-ether).

8. The process of claim 7, wherein methanol is added to the solution of clopidogrel base prior to combining the solution of Clopidogrel base with the sulfuric acid.

9. The process of claim 7, wherein Clopidogrel base is first combined with MTBE and thereafter methanol is added.

10. The process of claim 7, wherein the solution of Clopidogrel base in MTBE is added to the sulfuric acid.

11. The process of claim 1, further comprising isolating the clopidogrel Bisulphate Form I.

12. The process of claim 1, wherein the sulfuric acid is added at a temperature of about −20° C. to about 40° C.

13. The process of claim 12, wherein the sulfuric acid is added at a temperature of about −10° C. to about 0° C.

14. The process claim 1, wherein the sulfuric acid is added in a period of time of about 0.5 hours to about 5 hours.

15. The process of claim 1, wherein a suspension comprising Clopidogrel Bisulphate salt is obtained after addition of sulfuric acid, and wherein such suspension is stirred for about 1 hour to about 70 hours before isolating.

16. The process of claim 15, wherein the suspension is stirred for about 4 hours to about 24 hours.

17. The process of claim 1, wherein Clopidogrel Bisulphate Form I is further isolated by filtration.

18. The process of claim 17, wherein the filtration is carried out under a temperature of about −10° C. to about 30° C.

19. The process of claim 18, wherein the filtration is carried out under a temperature of about 10° C. to about 30° C.

20. The process of claim 11, further comprising drying the isolated clopidogrel Bisulphate Form I.

21. The process of claim 20, wherein drying is carried out under vacuum and at a temperature of about 30° C. to about 40° C.,

22. The process of claim 1, wherein the clopidogrel base is prepared by dissolving Clopidogrel Camphorsulphonate salt in a mixture of water and MIBK (methyl-isobutyl ketone) to obtain a solution and adding a base to the solution.

23. The process of claim 22, wherein the base is an alkali metal/alkaline earth metal hydroxide or carbonate, preferably sodium or potassium hydroxide.

24. The process of claim 22, wherein sodium or potassium hydroxide is added to the solution to obtain a pH of about 2-3 followed by addition of NaHCO₃ to obtain a pH of about 8.

25. The process of claims 22, wherein the reaction mixture is cooled during addition of the base to maintain a temperature of about 25 to about 30° C.

26. The process of claims 22, wherein a two phase reaction mixture comprising an organic phase is obtained and the organic phase is separated and washed with water.

27. The process of claim 26, further comprising concentrating the organic phase.

28. The process of claim 27, further comprising distilling the organic phase to remove water.

29. The process of claim 1, further comprising adding a surfactant before precipitation of clopidogrel bisulphate Form I.

30. A process for preparing clopidogrel Bisulphate Form I comprising dissolving Clopidogrel base in MTBE (methyl-t-butyl-ether); cooling; adding formic acid or acetic acid to obtain a cooled solution; and adding the cooled solution to a mixture of sulfuric acid and MTBE at a temperature less than about 40° C. to obtain Clopidogrel Bisulphate.

31. The process of claim 30 wherein the solution of Clopidogrel base and MTBE is cooled to a temperature of about −10° C. to about 0° C.

32. A process for preparing clopidogrel Bisulphate Form I comprising combining Clopidogrel Bisulphate, MIBK (methyl iso-butyl ketone) and clopidogrel base to obtain a suspension, and adding H₂SO₄ to the suspension, wherein the process is at a temperature of about 10° C. to about −20° C. and the Clopidogrel Bisulphate is an amount of at least about 10% weight/weight from the obtained Clopidogrel Bisulphate.

33. The process of claim 32, wherein the temperature is about −10° C.

34. The process of claim 1, wherein the organic solvent is MIBK and wherein the Clopidogrel Bisulphate and MIBK are first combined to obtain a suspension and Clopidogrel base dissolved in MIBK is then added to the suspension.

35. A process for preparing clopidogrel Bisulphate Form I comprising combining clopidogrel base, MIBK (methyl iso-butyl ketone) and surfactant to obtain a solution, and adding H₂SO₄, wherein the process is at a temperature of about 15° C. to about −15° C.

36. The process of claim 35, wherein the process is at a temperature of about 5° C.

37. The process of claim 35, wherein the surfactant is selected from the group consisting of polysorbate and Sodium Lauryl Sulfate (SLS).

38. The process of claim 30, wherein the MTBE is MIBK and wherein the Clopidogrel Bisulphate and MIBK are first combined to obtain a suspension and Clopidogrel base dissolved in MIBK is then added to the suspension.

39. The process of claim 32, wherein the Clopidogrel Bisulphate and MIBK are first combined to obtain a suspension and Clopidogrel base dissolved in MIBK is then added to the suspension.
